(19) United States
(12) Patent Application Publication
(10) Pub. No.: US 2012/0130266 A1
(43) Pub. Date: May 24, 2012

(54) COGNITIVE EFFICACY ESTIMATION SYSTEM AND METHOD

(75) Inventors: Santosh Mathan, Seattle, WA (US); Patricia May Ververs, Ellicott City, MD (US); Andrew Smart, Minneapolis, MN (US)

(73) Assignee: HONEYWELL INTERNATIONAL INC., Morristown, NJ (US)

(21) Appl. No.: 12/953,763

(22) Filed: Nov. 24, 2010

(51) Int. Cl. A61B 5/0476

(52) U.S. Cl. 600/544

(57) ABSTRACT

A system and method for estimating cognitive efficacy of an individual is provided. The individual is subjected to a plurality of tasks. Electroencephalogram (EEG) data from EEG sensors coupled to the individual are collected while the individual is performing the plurality of tasks. The EEG data are supplied to a trained classifier that generates an estimate of cognitive effort of the individual for each of the plurality of tasks performed by the individual.

### TECHNICAL FIELD

The present invention generally relates to cognitive efficacy estimation, and more particularly relates to a system and method for estimating the change in cognitive efficacy of humans following a moderate traumatic brain injury.

### BACKGROUND

Brain injuries can occur in many ways. A mild traumatic brain injury (MTBI), commonly referred to as concussion, can result from any one of numerous head-strike events. Such events may include blast exposure, vehicle accidents, falls, or blows from other people. While the symptoms of MTBI are relatively mild and transient for most individuals, for some individuals cognitive deficits can persist for years. Other brain injuries, such as those caused by insufficient oxygen, poisoning, or infection, can cause similar deficits.

Measures that permit inferences about underlying cognitive efficacy can play a vital role in identifying individuals with MTBI-related cognitive impairment. Such measures also serve an important function in tracking individual progress over the course of rehabilitation. However, widely used measures, such as behavioral measures and self-report measures, are oftentimes limited in terms of insight. For example, researchers have noted that behavioral measures often do not provide a sufficient basis for discriminating between fluid performance and effortful performance stemming from compromised cognitive function. Furthermore, behavioral measures are often compared to group norms to identify injury related cognitive deficit. While this may help identify individuals with pronounced cognitive deficits, individuals with MTBI may experience personally significant changes in cognitive function that may lie within the range expected among uninjured individuals. Self-report measures suffer drawbacks associated with retrospection and subjectivity. These measures are also summative in nature, and may not capture fluctuations in cognitive effort in dynamic task environments.

Other tools currently used to diagnose and rehabilitate individuals with MTBI also suffer drawbacks. For example, structural imaging techniques often do not reveal diffuse microscopic injuries associated with MTBI. Traditional neuropsychological tests are often insensitive to deficits that may become apparent in operational task contexts. Without a precise and timely estimate of cognitive efficacy, it may be hard to structure an appropriate course of rehabilitation, and to assess progress. At best, these limitations can make rehabilitation inefficient; at worst, these limitations can render efforts ineffective.

Hence, there is a need for a system and method that provides objective, fine-grained, and automated estimates of cognitive function in a variety of task contexts so that the progress of individuals with MTBI can be accurately assessed over the course of rehabilitation and/or post-injury cognitive impairment can be assessed and rehabilitation efforts can be tailored to individual strengths and weaknesses. The present invention addresses at least these needs.

### BRIEF SUMMARY

In one embodiment, a method for estimating cognitive efficacy of an individual includes collecting electroencephalogram (EEG) data from EEG sensors coupled to the individual, while the individual is performing a plurality of tasks. The EEG data are supplied to a trained classifier that generates an estimate of cognitive effort of the individual for each of the plurality of tasks performed by the individual. Baseline cognitive effort data for the individual are generated based on the plurality of tasks, and the estimates of cognitive effort are compared to the baseline cognitive effort data to determine a change in cognitive efficacy of the individual.

In another embodiment, a system for estimating cognitive efficacy of an individual performing a plurality of tasks includes a plurality of electroencephalogram (EEG) sensors, memory, and a processor. The EEG sensors are adapted to be coupled to the individual, and each EEG sensor is configured to collect EEG data from the individual, while the individual is performing the plurality of tasks, and supply the collected EEG data. The memory has baseline cognitive effort data for the individual stored therein. The baseline cognitive effort data being based on the plurality of tasks. The processor is in operable communication with the memory and is coupled to receive the collected EEG data. The processor is configured, upon receipt of the EEG data, to generate an estimate of cognitive effort of the individual for each of the plurality of tasks performed by the individual, selectively retrieve the baseline cognitive effort data from the memory, and compare the estimate of cognitive effort to the baseline cognitive effort data to determine a change in cognitive efficacy of the individual.

In yet another embodiment, a system for estimating cognitive efficacy of an individual performing a plurality of tasks includes a plurality of EEG sensors, memory, and a processor. The EEG sensors are adapted to be coupled to the individual, and each EEG sensor is configured to collect EEG data from the individual, while the individual is performing the plurality of tasks, and supply the collected EEG data. The memory has baseline cognitive effort data for the individual stored therein. The baseline cognitive effort data being based on the plurality of tasks. The processor is configured to implement a trained classifier, is in operable communication with the memory, and is coupled to receive the collected EEG data. The processor is further configured, upon receipt of the EEG data, to generate an estimate of cognitive effort of the individual for each of the plurality of tasks performed by the individual, selectively retrieve the baseline cognitive effort data from the memory, compare the estimate of cognitive effort to the baseline cognitive effort data to determine a change in cognitive efficacy of the individual, and compare updated estimates of cognitive effort to the baseline cognitive effort data to track changes in the cognitive efficacy of the individual.

Furthermore, other desirable features and characteristics of the cognitive efficacy estimation system and method will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and wherein:

FIG. 1 depicts a functional block diagram of a system for estimating cognitive efficacy of an individual;

FIG. 2 depicts an embodiment of a wireless electroencephalogram (EEG) sensor cap that may be used to implement the system of FIG. 1;

FIGS. 3A-3C and FIG. 4 illustrate different methods of artifact noise removal from EEG data;

FIG. 5 depicts the logistic function; and

FIG. 6 depicts an exemplary process, in flowchart form, that may be implemented by the system of FIG. 1.

### DETAILED DESCRIPTION

The following detailed description is merely exemplary in nature and is not intended to limit the invention or the application and uses of the invention. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention which is defined by the claims.

Furthermore, there is no intention to be bound by any expressed or implied theory presented in the preceding technical field, background, brief summary, or the following detailed description. In this regard, the inventive concepts described herein may be applied in many contexts, including military, athletic, and medical contexts.

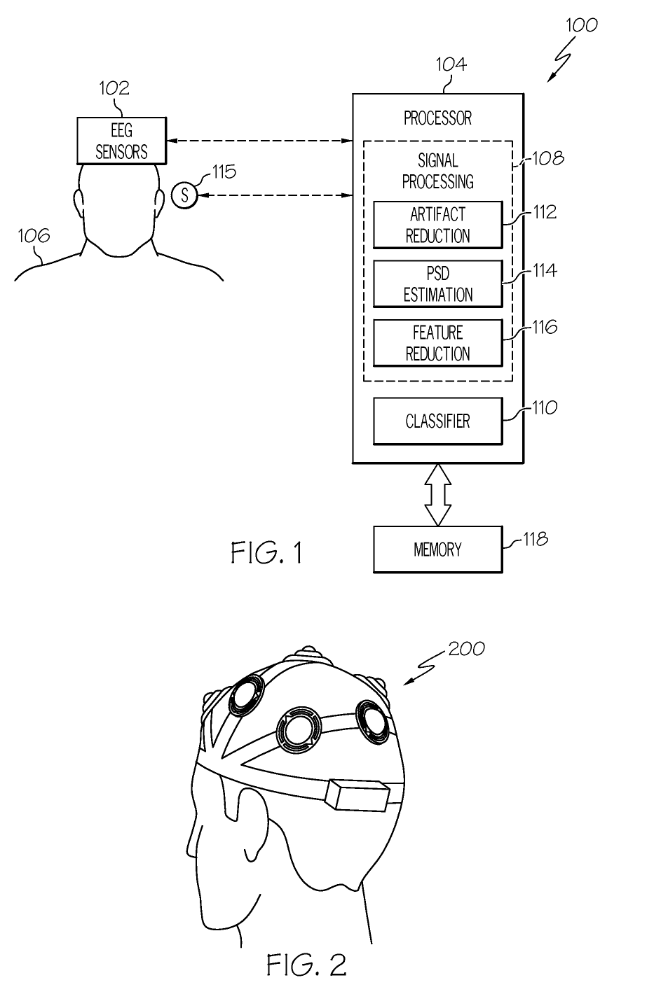

Turning now to FIG. 1, a functional block diagram of a system for estimating cognitive efficacy of an individual is depicted, and includes a plurality of electroencephalogram (EEG) sensors 102 and a processor 104. The EEG sensors 102 are preferably implemented using a 64-channel, wireless EEG sensor cap that is worn by an individual 106. An embodiment of a suitable wireless EEG sensor cap 200 is depicted in FIG. 2. The use of a wireless EEG sensor cap 200, such as the one depicted in FIG. 2, makes it possible to collect high-quality EEG data outside the constraints of the laboratory environment. The depicted wireless EEG sensor cap is also relatively easy to set up, and is relatively comfortable for the individual to wear over long periods of time. No matter the specific implementation, each EEG sensor 102 is configured to collect EEG data from the individual 106, and supply the collected EEG data to the processor 104. As will be described in more detail further below, the EEG data are preferably collected while the individual 106 is performing a plurality of tasks.

The processor 104 is coupled to receive the collected EEG data and is configured, upon receipt of the EEG data, to generate an estimate of the cognitive effort of the individual 106 while the individual 106 is performing the plurality of tasks. To implement this functionality, the processor 104, at least in the depicted embodiment, is configured to implement various signal processing functions 108, and a cognitive state trained classifier 110. It will be appreciated that in some embodiments one or more of the signal processing functions 108 could be implemented, if need or desired, using analog and/or digital circuits/devices that are physically separate from the processor 104.

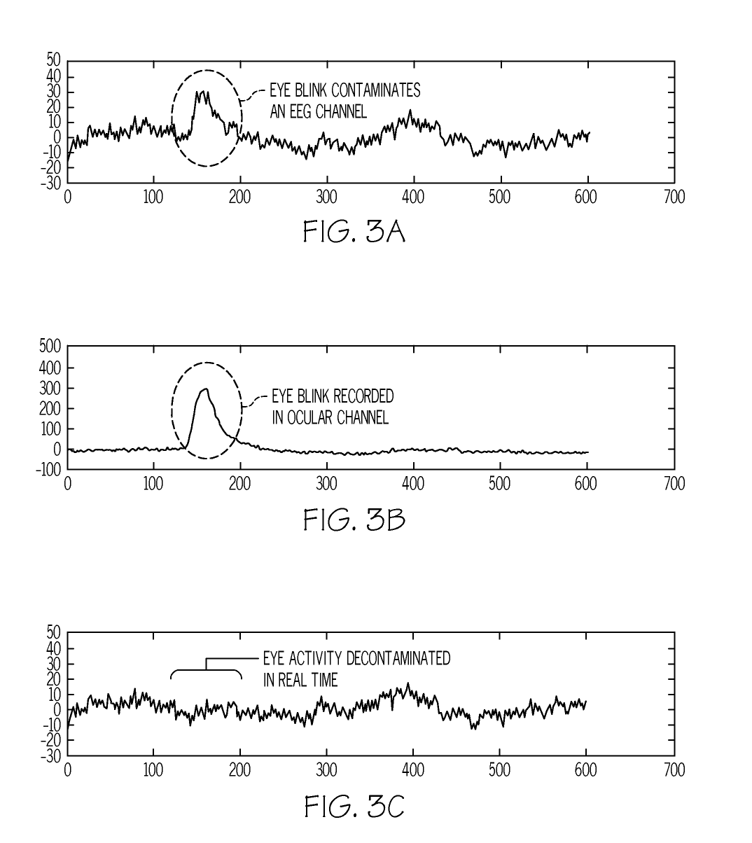

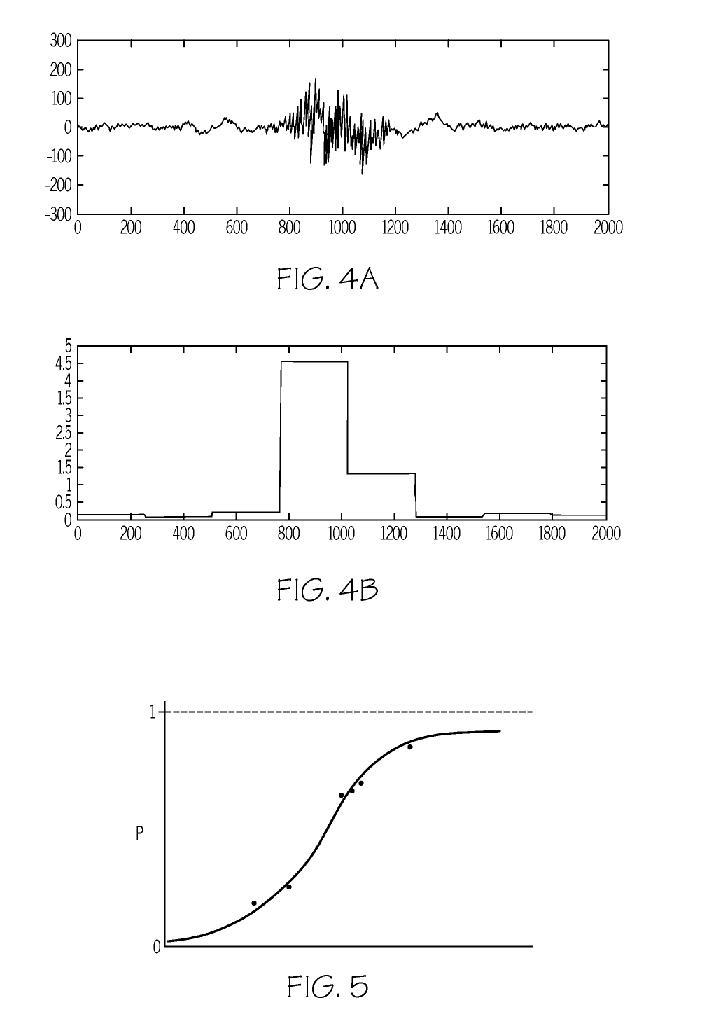

The data gathered by the EEG sensors 102 may be contaminated with noise artifacts, and may additionally contain information of little use for estimating cognitive effort. Hence, the signal processing functions 108 implemented by the processor 104 preferably include artifact reduction processing 112, power spectral density estimation processing 114, and feature reduction processing 116. The artifact reduction processing 112 will vary depending on the nature of the artifacts. For example, some artifacts, such as DC drift and electrical line noise, lie outside the frequency bands that are typically relevant for cognitive state estimation. These types of artifacts are rejected using digital filters. Other types of artifacts, such as eye blinks and noise related to muscle activity, overlap with the frequency bands of relevance for cognitive state estimation. Some of these artifacts can be measured precisely at the source, whereas others cannot. For example, eye blinks can be precisely and reliably measured using sensors 115 (which may be wired or wireless), but some muscle activity cannot. For those artifact sources that can be precisely and reliably measured, adaptive linear filters may be used to estimate the propagation of noise activity to individual EEG sites. This information is then used to subtract the contribution of noise activity from each site. An example of how this may be implemented is illustrated in FIGS. 3A-3C. For those artifacts that cannot be precisely and reliably measured, data samples contaminated by these types of artifacts can be identified and rejected. An example of how this may be implemented is illustrated in FIG. 4.

Following artifact detection and reduction, the power spectral density estimation processing 114 spectrally decomposes EEG data, and estimates the EEG power at various frequency bands. The frequency bands are 4-8 Hz (theta), 8-12 Hz (alpha), 13-30 Hz (beta), and 30-40 Hz (gamma). These estimates form the features that are the basis for estimating cognitive state. However, before being supplied to the trained classifier 110, the feature reduction processing 114 normalizes the EEG sensor data so that any differences in the dynamic range of various features do not bias the trained classifier 110.

The feature reduction processing 114 implements dimensionality reduction techniques to eliminate potentially uninformative features in the data. The specific techniques that are implemented may vary, and include principal component analysis (PCA) and Fisher discriminant analysis, just to name two. No matter the specific technique that is used, the analysis identifies and retains features that account for most of the variance in the EEG data or contain information critical for discriminating between states of interest.

The trained classifier 110 uses statistical machine learning principles to automatically classify individual cognitive states in real-time. In particular, the trained classifier 110 is configured to generate an estimate of the cognitive effort of the individual 106 as a value that lies between 0 and 1, where 0 represents relatively low cognitive effort and 1 represents relatively high cognitive effort. Although various classification algorithms may be used to provide this functionality, the trained classifier 110, at least in the depicted embodiment, implements the logistic regression model. The logistic regression model assumes the class conditional probability will follow the logistic function:

$$f = p 〈 c \vert x 〉 = \frac{{e}^{y}}{1 + {e}^{y}} = \frac{{e}^{{w}^{T} x + b}}{1 + {e}^{{w}^{T} x + b}} ,$$

which is parameterized by the weight vector “w” and bias “b.” The parameters are adjusted by maximizing the likelihood of the data so that the data matches the logistic function delineated above and illustrated in FIG. 5.

Before proceeding further, it is noted that differences among individuals can produce different physical and neurophysiological responses to task demands. Consequently, the trained classifier 110 is individualized so that it performs better than a trained classifier that is based on group norms. To individualize the trained classifier 110, it is constructed using baseline cognitive effort data that are collected while the individual 106 is carrying out tasks in the individual's unique task environment. Moreover, in order to gather the baseline cognitive effort data, the cognitive demands imposed by the task environment are explicitly manipulated to represent the extremes of cognitive load that is to be expected in the task environment. The baseline cognitive effort data that are collected while the individual 110 performs various tasks in this environment are used as the basis for constructing the trained classifier 110. These baseline cognitive effort data are preferably stored in a memory 118 that is in operable communication with the processor 104. As will be described further below, the baseline cognitive effort data may also be used to determine changes in the cognitive efficacy of the individual 106.

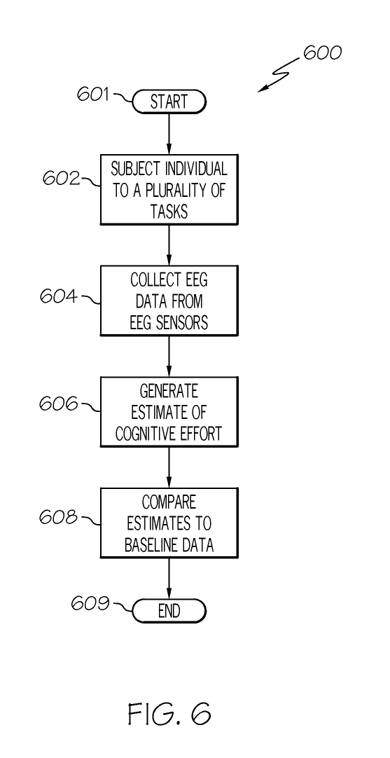

As was previously noted, the processor 104 receives the EEG data and generates an estimate of the cognitive effort of the individual 106 for each of a plurality of tasks performed by the individual 106. The overall process 600 by which the processor 104 implements this outcome is depicted in flowchart form in FIG. 6, and with reference thereto will now be described in more detail. Before doing so, however, it is noted that the depicted process 600 is merely exemplary of any one of numerous ways of depicting and implementing the overall process to be described. Moreover, before the process 600 is initiated, it is noted that the EEG sensors 102 have preferably been properly applied to the individual 106, and appropriately configured to collect EEG data. It is additionally noted that the numerical parenthetical references in the following description refer to like steps in the flowchart depicted in FIG. 6.

The process 600 begins by subjecting the individual 106 to a plurality of tasks (602). The tasks are preferably varied with respect to the relative level of cognitive effort needed to complete the task, and are preferably tailored to the individual's task environment. For example, if the individual is a soldier, the tasks may include one or more of vehicle driving, firearm shooting, field-based battle scenarios, each at varying levels of difficulty. If the individual is a pilot, the tasks may include various aircraft manipulation procedures at varying levels of difficulty, and so on.

While the individual 106 is performing the tasks, EEG data are collected from the EEG sensors 102 and are supplied to the processor 104 (604). The processor 104, and more specifically the trained classifier 110 implemented in the processor 104, generates an estimate of the cognitive effort of the individual 106 for each of the plurality of tasks performed by the individual 106 (606). The estimates of cognitive effort may then be compared to the baseline cognitive effort data stored in memory 118 to determine a change in the cognitive efficacy of the individual 106 (608). It will be appreciated that the estimates of cognitive effort may be periodically compared to the baseline data to determine changes in the cognitive efficacy of the individual.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps. However, it should be appreciated that such block components (or modules) may be realized by any number of hardware, software, and/or firmware components configured to perform the specified functions. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments described herein are merely exemplary implementations.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for estimating cognitive efficacy of an individual, comprising the steps of:
collecting electroencephalogram (EEG) data from EEG sensors coupled to the individual, while the individual is performing a plurality of tasks;
supplying the EEG data to a trained classifier that generates an estimate of cognitive effort of the individual for each of the plurality of tasks performed by the individual;
generating baseline cognitive effort data for the individual, based on the plurality of tasks; and
comparing the estimates of cognitive effort to the baseline cognitive effort data to determine a change in cognitive efficacy of the individual.

2. The method of claim 1, further comprising:
preprocessing the EEG data; and
supplying the preprocessed EEG data to the trained classifier.

3. The method of claim 2, wherein the step of preprocessing comprises:
reducing artifacts in the EEG data; and
reducing non-informative features in the EEG data.

4. The method of claim 3, wherein the step of reducing artifacts in the EEG data comprises:
filtering the EEG data;
measuring artifacts at one or more artifact source; and
removing artifacts in the EEG data that coincide with the measured artifacts.

5. The method of claim 3, wherein the step of reducing non-informative features in the EEG data comprises subjecting the EEG data to one or more dimensionality reduction techniques.

6. The method of claim 1, wherein the trained classifier is configured to implement a logistic regression technique that follows a logistic model.

7. The method of claim 1, wherein the estimate of cognitive effort is a value between 0 and 1.

8. The method of claim 7, wherein 0 represents relatively low cognitive effort and 1 represents relatively high cognitive effort.

9. The method of claim 1, further comprising:
comparing updated estimates of cognitive effort to the baseline cognitive effort data to track changes in the cognitive efficacy of the individual.

10. A system for estimating cognitive efficacy of an individual performing a plurality of tasks, the system comprising:
a plurality of EEG sensors adapted to be coupled to the individual, each EEG sensor configured to collect EEG data from the individual, while the individual is performing the plurality of tasks, and supply the collected EEG data;
memory having baseline cognitive effort data for the individual stored therein, the baseline cognitive effort data based on the plurality of tasks; and
a processor in operable communication with the memory and coupled to receive the collected EEG data and configured, upon receipt of the EEG data, to (i) generate an estimate of cognitive effort of the individual for each of the plurality of tasks performed by the individual, (ii) selectively retrieve the baseline cognitive effort data from the memory, and (iii) compare the estimate of cognitive effort to the baseline cognitive effort data to determine a change in cognitive efficacy of the individual.

11. The system of claim 10, wherein the processor is configured to implement a trained classifier that generates the estimate of cognitive effort.

12. The system of claim 11, wherein the trained classifier is configured to implement a logistic regression technique that follows a logistic model:

13. The system of claim 10, wherein the estimate of cognitive effort is a value between 0 and 1.

14. The method of claim 13, wherein 0 represents relatively low cognitive effort and 1 represents relatively high cognitive effort.

15. The system of claim 10, further comprising:
one or more artifact sensors configured to measure artifacts at one or more artifact sources and supply artifact data representative thereof,
wherein the processor is further coupled to receive the artifact data, and is further configured to remove artifacts in the EEG data that coincide with the measured artifacts.

16. The system of claim 10, wherein the processor is further configured to implement one or more dimensionality reduction techniques to remove undesired features from the EEG data.

17. The system of claim 10, wherein the processor is further configured to compare updated estimates of cognitive effort to the baseline cognitive effort data to track changes in the cognitive efficacy of the individual.

18. A system for estimating cognitive efficacy of an individual performing a plurality of tasks, the system comprising:
a plurality of EEG sensors adapted to be coupled to the individual, each EEG sensor configured to collect EEG data from the individual, while the individual is performing the plurality of tasks, and supply the collected EEG data;
memory having baseline cognitive effort data for the individual stored therein, the baseline cognitive effort data based on the plurality of tasks; and
a processor configured to implement a trained classifier, the processor in operable communication with the memory and coupled to receive the collected EEG data and further configured, upon receipt of the EEG data, to:
generate an estimate of cognitive effort of the individual for each of the plurality of tasks performed by the individual,
selectively retrieve the baseline cognitive effort data from the memory,
compare the estimate of cognitive effort to the baseline cognitive effort data to determine a change in cognitive efficacy of the individual, and
compare updated estimates of cognitive effort to the baseline cognitive effort data to track changes in the cognitive efficacy of the individual.

19. The system of claim 18, wherein the trained classifier is configured to implement a logistic regression technique that follows a logistic model:

20. The system of claim 19, wherein the estimate of cognitive effort is a value between 0 and 1.
